(19) United States
(12) Patent Application Publication
(10) Pub. No.: US 2025/0090227 A1
(43) Pub. Date: Mar. 20, 2025

(54) PORTABLE DEPILATION INSTRUMENT

(71) Applicant: SHENZHEN ULIKE SMART ELECTRONICS CO., LTD., SHENZHEN (CN)

(72) Inventor: YUPING PAN, SHENZHEN (CN)

(21) Appl. No.: 18/969,236

(22) Filed: Dec. 4, 2024

Related U.S. Application Data
(63) Continuation of application No. 17/597,476, filed on Jan. 7, 2022, now Pat. No. 12,207,868.

(30) Foreign Application Priority Data
Jul. 7, 2019 (CN) 201921052794.0
Sep. 4, 2019 (CN) 201921467936.X
Nov. 18, 2019 (CN) 201921999189.4

(51) Int. Cl. A61B 18/18; A61B 18/00

(52) U.S. Cl. CPC A61B 18/18; A61B 2018/00017; A61B 2018/00476

(57) ABSTRACT

A portable depilation instrument includes a shell, a cold compress portion, and an emitter. The shell defines an accommodating space. The cold compress portion is exposed from the shell and configured to contact skin of a user. The emitter is arranged in the accommodating space and located on a side of the cold compress portion facing away from the skin of the user, and the emitter is configured to emit light that irradiates the skin of the user. The portable depilation instrument defines a first channel and a second channel inside the shell, allowing external cooling medium entering the shell to separately flow through the first channel to cool the emitter and flow through the second channel to cool the cold compress portion.

### CROSS REFERENCE TO RELATED APPLICATION

The present disclosure is a continuation of application Ser. No. 17/597,476, filed on Jan. 7, 2022.

### TECHNICAL FIELD

The present invention relates to the field of depilation and beauty, and specifically, to a portable depilation instrument.

### BACKGROUND

A light depilation technology is a non-invasive modern depilation technology, in which unwanted hair can be safely, quickly, and efficiently removed from a body. Specifically, light depilation is based on a theory of selective photothermolysis, in which a specific wavelength of light is allowed to penetrate epidermis without damaging epidermal hair follicles, and melanin in a hair shaft selectively absorbs light energy, so that the hair follicles are coagulated and necrotic during heating, to effectively slow down growth, thereby implementing depilation.

However, it is well known that all light generates a lot of heat during use, which brings a burning pain to skin, especially a household portable depilation instrument. To help users hold and use, all the portable depilation instruments are designed relatively small, and therefore, it is easier to form heat accumulation. In this case, if heat dissipation and temperature reduction cannot be implemented in time, a relatively strong pain and a redness and swollen phenomenon are brought to the users, and even a security problem is caused.

Therefore, in the prior art, to reduce heat generated when the portable depilation instrument performs depilation, a sealing member is disposed in a heat dissipation structure, and an opening is provided on the sealing member and is in communication with outside, to match a heat dissipation fin to independently form a channel for a heating member that needs to be cooled to perform heat dissipation, thereby effectively improving the effect of heat dissipation and temperature reduction. However, the portable depilation instrument has more than one element that generates heat, and generally, internal circuits and other elements also generate heat. Consequently, how to make all the elements that generate heat dissipate the heat under a same power, and how to improve the cooling effect and maximize the efficiency are problems to be resolved currently.

### SUMMARY

To resolve a problem of incomplete temperature reduction of an existing portable depilation instrument, the present invention provides a portable depilation instrument.

To resolve the foregoing technical problem, the present invention provides a technical solution as follows: a portable depilation instrument, including a shell, defining an accommodating space; a cold compress portion, exposed from the shell and configured to contact skin of a user; and an emitter, arranged in the accommodating space and located on a side of the cold compress portion facing away from the skin of the user, and the emitter being configured to emit light that irradiates the skin of the user. The portable depilation instrument defines a first channel and a second channel inside the shell, allowing external cooling medium entering the shell to separately flow through the first channel to cool the emitter and flow through the second channel to cool the cold compress portion.

### BRIEF DESCRIPTION OF THE DRAWINGS

FIG. 1 is a schematic structural diagram of a portable depilation instrument according to a first embodiment of the present invention;

FIG. 2 is a schematic exploded view of the portable depilation instrument according to the first embodiment of the present invention;

FIG. 3 is a first partial exploded view of a depilation apparatus in the portable depilation instrument according to the first embodiment of the present invention;

FIG. 4 is a second partial exploded view of a depilation apparatus in the portable depilation instrument according to the first embodiment of the present invention;

FIG. 5 is a first partial exploded view of a fan in the portable depilation instrument according to the first embodiment of the present invention;

FIG. 6 is an exploded view of a heat conductive component in the portable depilation instrument according to the first embodiment of the present invention;

FIG. 7 is a first partial exploded view of a depilation mechanism in the portable depilation instrument according to the first embodiment of the present invention;

FIG. 8 is a third partial exploded view of the depilation apparatus in the portable depilation instrument according to the first embodiment of the present invention;

FIG. 9 is a second partial exploded view of the depilation mechanism in the portable depilation instrument according to the first embodiment of the present invention;

FIG. 10 is a cross-sectional view of the depilation mechanism in the portable depilation instrument according to the first embodiment of the present invention;

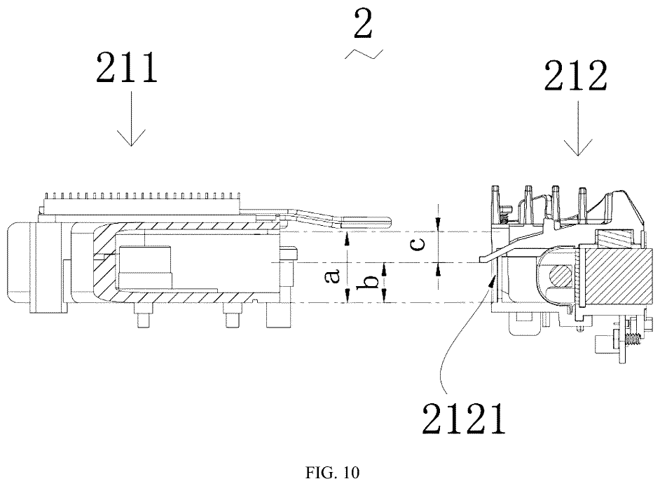

FIG. 11 is a fourth partial exploded view of the depilation apparatus in the portable depilation instrument according to the first embodiment of the present invention;

FIG. 12 is an assembled diagram of the depilation apparatus in the portable depilation instrument according to the first embodiment of the present invention;

FIG. 13 is a cross-sectional view of the depilation apparatus in the portable depilation instrument according to the first embodiment of the present invention;

FIG. 14 is a partially omitted view of the portable depilation instrument according to the first embodiment of the present invention;

FIG. 15 is a partial exploded view of the portable depilation instrument according to the first embodiment of the present invention;

FIG. 16 is a schematic structural diagram of a portable depilation instrument according to a second embodiment of the present invention;

FIG. 17 is an expanded view of a part structure of the portable depilation instrument according to the second embodiment of the present invention;

FIG. 18 is a schematic structural diagram of a heat conductive component in the portable depilation instrument according to the second embodiment of the present invention; and

FIG. 19 is a schematic structural diagram of a depilation apparatus in the portable depilation instrument according to the second embodiment of the present invention.

### DESCRIPTION OF REFERENCE NUMERALS

100. portable depilation instrument;

1. shell; 101. upper shell; 102. lower shell;

11. inlet; 12. head portion; 13. holding portion; 14. second outlet; 15. first outlet; 16. third outlet; 1601. first groove; 1602. second groove; 1603. exhaust structure;

2. depilation apparatus; 21. heat dissipation mechanism;

211. fan; 2111. fan air inlet; 2112. fan air outlet; 2113. heat conductive element; 2114. first heat dissipation fin;

212. heat conductive component; 2121. first vent; 2122. second vent; 2123. second heat dissipation fin; 2124. third vent;

22. depilation mechanism; 221. cold compress portion; 222. emitter; 223. refrigeration element; 2231. heating surface; 2232. refrigeration surface; 224. skin detection portion; 225. reflecting plate; 226. insulating plate;

3. circuit apparatus; and 4. power supply apparatus.

### DETAILED DESCRIPTION OF THE EMBODIMENTS

To make the objectives, technical solutions, and advantages of the present invention clearer and more comprehensible, the following further describes the present invention in detail with reference to the accompanying drawings and embodiments. It should be understood that specific embodiments described herein are only intended to explain the present invention, but are not intended to limit the present invention.

### Embodiment 1

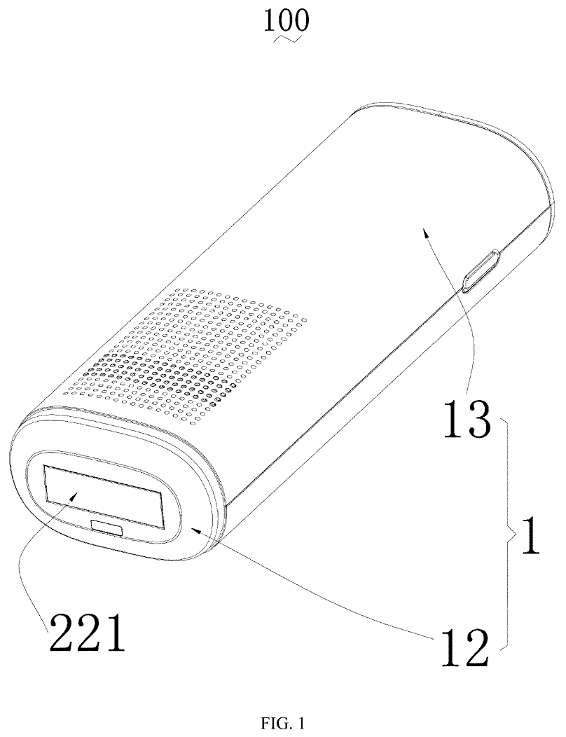

Referring to FIG. 1, the present invention provides a portable depilation instrument 100, configured to remove unwanted hair from a human body, and including a shell 1 and a cold compress portion 221 having a light-transmitting and cold compress effect. A user may tightly attach the cold compress portion 221 to a human skin surface, the portable depilation instrument 100 emits light, and the light passes through the cold compress portion 221 and penetrates the skin surface to reach a root of a hair follicle, so that the hair follicle is coagulated and necrotic, to remove hair.

The shell 1 mainly plays a role of protecting internal elements, and an overall shape of the shell is not limited, may be U-shaped, T-shaped, shuttle-shaped, or long strip-shaped, or is optionally long striped cylindrical.

The shell 1 includes a holding portion 13 and a head portion 12, the head portion 12 is at one end facing human skin, and the holding portion 13 is parallel with the head portion 12. After the head portion 12 is fixedly connected to the holding portion 13, an accommodating space is formed inside, and the cold compress portion 221 is exposed in the accommodating space from the head portion 12, to be used as a carrier for emitting light and contacting the human skin. The holding portion 13 is used for holding the portable depilation instrument 100, to facilitate operations of the user.

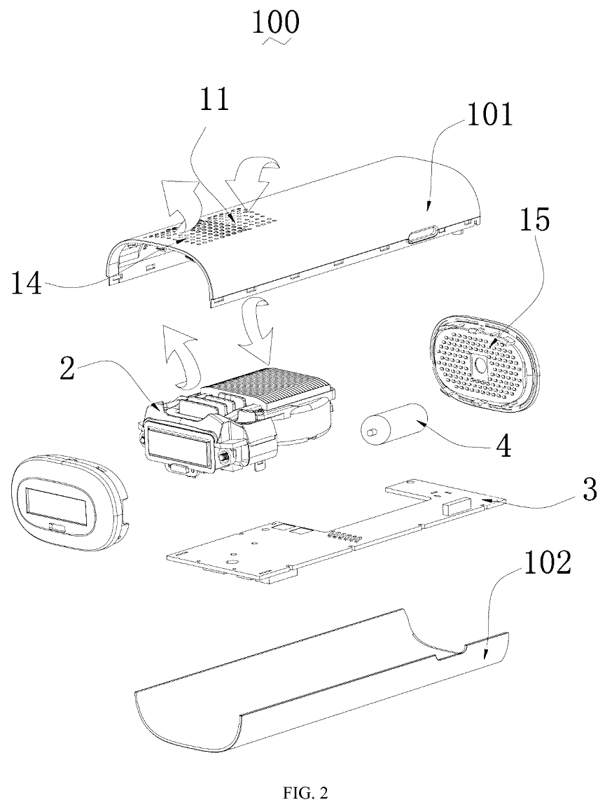

Referring to FIG. 2, the shell 1 includes an upper shell 101 and a lower shell 102, may be formed by assembling the upper shell 101 and the lower shell 102 through screw connection or clamping connection, or may be formed through integral processing, or is optionally formed through assembly in a clamping manner, to facilitate disassembly and assembly.

A depilation apparatus 2, a circuit apparatus 3, and a power supply apparatus 4 are included inside the accommodating space of the shell 1. The depilation apparatus 2, the circuit apparatus 3, and the power supply apparatus 4 are all sequentially electrically connected. The power supply apparatus 4 may provide electric energy to the depilation apparatus 2, so that the depilation apparatus can be used without plugging in an external circuit, or can be used by directly using an external power source, such as a dry battery or an energy storage battery. Therefore, the depilation apparatus is convenient to store and carry, and can be used outside. The circuit apparatus 3 may be externally connected to an external circuit to charge the power supply apparatus 4 and control the entire portable depilation instrument 100 on/off, power regulation, and temperature protection.

A person skilled in the art may understand that, during working, all elements in the depilation apparatus 2, the circuit apparatus 3, and the power supply apparatus 4 generate heat.

In daily production, some control elements (not shown in the figure) in the circuit apparatus 3 are generally disposed on a surface of the shell 1, to form a switch, a screen, and an operation panel. The user may control the entire portable depilation instrument 100 through manipulation, to adjust the on/off of the portable depilation instrument 100 and control levels.

The depilation apparatus 2 is disposed at one end near the head portion 12 of the shell 1, and may be fixedly clamped inside the accommodating space of the shell 1. An inlet 11 and a second outlet 14 corresponding to a region of the depilation apparatus 2 are disposed on one side of the shell 1, that is, the upper shell 101 or the lower shell 102. It may be understood that, both the inlet 11 and the second outlet 14 are disposed on a same side of the shell 1, and are closely arranged together. This design is to save a space of the shell 1, so that the shell 1 can be relatively shorter, and is compact and precise, and the inlet 11 and the second outlet 14 are designed together to shorten an air circulation distance of air, to implement rapid air circulation, so that the heat dissipation efficiency is higher.

Further, the first outlet 15 is disposed at an end portion of the shell 1, that is, one end of the shell 1 away from the head portion 12. The reason why the first outlet 15 is remotely disposed is to increase a distance through which the air flows, so that a cooling range is wider.

It may be understood that, during normal production, both the circuit apparatus 3 and the power supply apparatus 4 are disposed inside the shell, and are optionally disposed inside the holding portion 13. In this embodiment, both the circuit apparatus and the power supply apparatus are disposed on a back side of the depilation apparatus 2, that is, one side of the depilation apparatus 2 away from the human skin. By using this design, a range of the air involved is expanded, to achieve an overall cooling effect inside the shell.

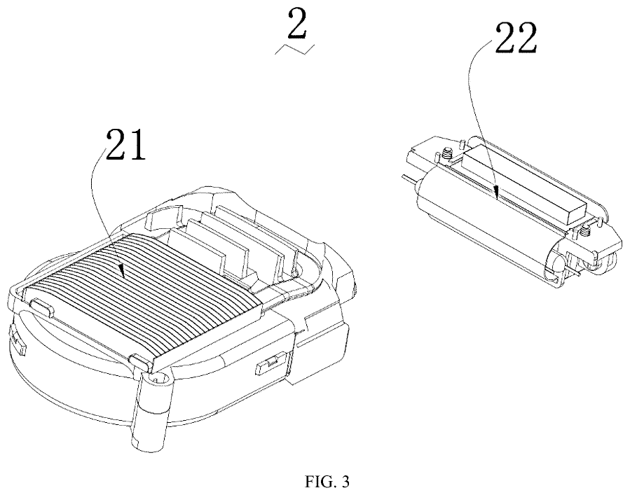

Referring to FIG. 3, the depilation apparatus 2 includes a heat dissipation mechanism 21 and a depilation mechanism 22. The depilation mechanism 22 emits light to remove hair from the human body, and is mainly in contact with the human skin. The heat dissipation mechanism 21 is used for reducing a temperature, so that the human body does not have a burning sensation under the light; and the heat dissipation mechanism 21 and the depilation mechanism 22 may be connected through clamping connection or screw connection, or are optionally connected through screw connection, so that fixation is more stable.

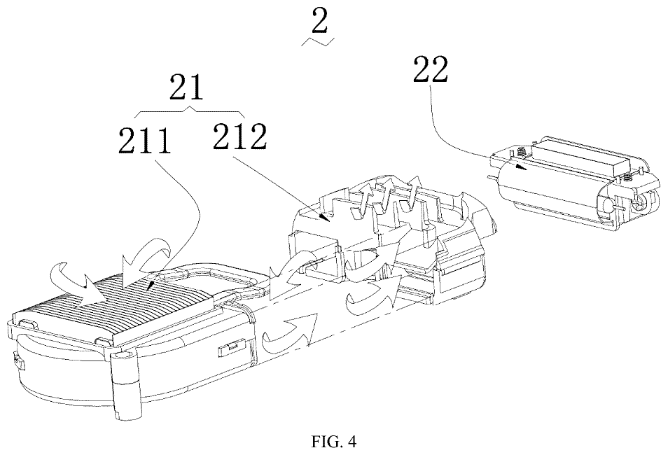

Referring to FIG. 4, the heat dissipation mechanism 21 includes a fan 211 and a heat conductive component 212. The fan 211 may be a centrifugal, axial-flow, mixed-flow, and cross-flow fan, or is optionally a centrifugal fan, so that external air mainly flows radially after axially entering an impeller of the fan. The heat conductive component 212 and the fan 211 may be connected through screw connection or clamping connection, so that air sucked by the fan 211 may flow through the heat conductive component 212 in a split direction. The depilation mechanism 22 is connected to one surface of the heat conductive component 212 away from the fan 211, and therefore, air flow in the heat conductive component 212 may perform cooling and temperature reduction on the demodulation mechanism 22, to achieve an ice compress effect on the human skin.

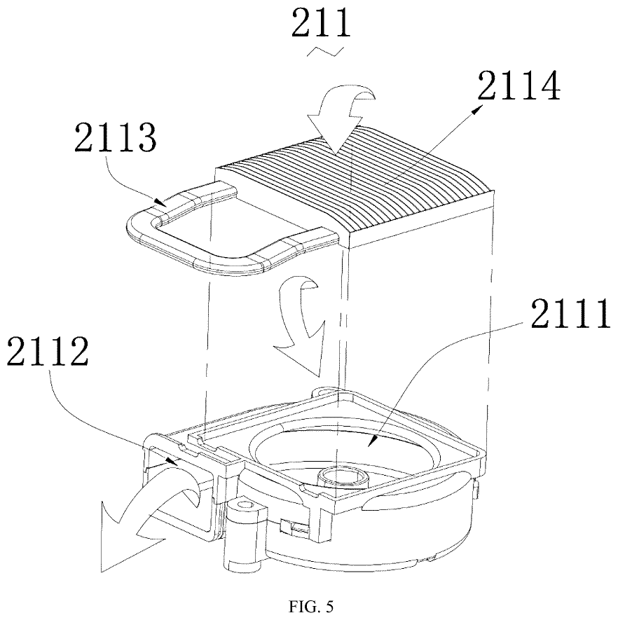

Referring to FIG. 1 and FIG. 5, a specific structure of the fan 211 is as follows:

The fan 211 includes a fan air inlet 2111 and a fan air outlet 2112. A first heat dissipation fin 2114 is disposed at the fan air inlet 2111, and the first heat dissipation fin 2114 is disposed at an end portion of the heat conductive element 2113. That is, the heat conductive element 2113 is located at one end near the fan air inlet 2111, and the fan 211 is disposed at one end of the fan air inlet 2111 facing the inlet 11 on the shell 1, to suck air through inside of the inlet 11 on the shell 1, where the first heat dissipation fin 2114 completely covers the fan air inlet 2111, so that before entering the fan air inlet 2111, the external air passes through the first heat dissipation fin 2114 for heat dissipation and temperature reduction for a first time. A portion of the heat conductive element 2113 is inserted into the first heat dissipation fin 2114, and a remaining portion extends outward.

Further, the first heat dissipation fin 2114 may be a cast iron heat dissipation fin, a steel heat dissipation fin, and or an aluminum alloy heat dissipation fin. It may be understood that, because the first heat dissipation fin 2114 dissipates heat in the form of convection, a larger coverage area indicates a better heat dissipation effect.

Further, the heat conductive element 2113 may be a heat conductive tube, and a material of the heat conductive element is selected as a sheet structure or a tubular structure made of a metal or an alloy such as silver, copper, or iron, or is optionally a sheet structure. Due to a unique metal heat transfer property, the heat conductive element 2113 may dissipate the heat integrally through the first heat dissipation fin 2114.

It may be understood that, in actual production, the first heat dissipation fin 2114 and the heat conductive element 2113 are often of an integrated structure, and the first heat dissipation fin 2114 and the heat conductive element 2113 are connected together. Under the action of metal heat transfer, as the fan 211 sucks air inward, the heat on the heat conductive element 2113 flows quickly, thereby implementing quick temperature reduction.

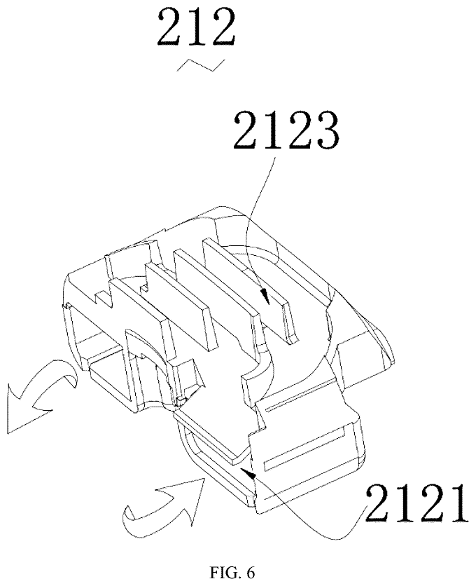

Referring to FIG. 6, the heat conductive component 212 includes a first vent 2121. The first vent 2121 is disposed at one end of the heat conductive component 212 facing the fan 211, or the first vent 2121 is optionally disposed parallel to one end of the heat conductive component 212, so that after entering through the first vent 2121 on one side, the external cooling medium is discharged in an opposite direction from the first vent 2121 on the other side. In addition, a second heat dissipation fin 2123 is disposed at a surface of one side of the heat conductive component 212, and the second heat dissipation fin 2123 is optionally disposed on an upper side of the heat conductive component 212.

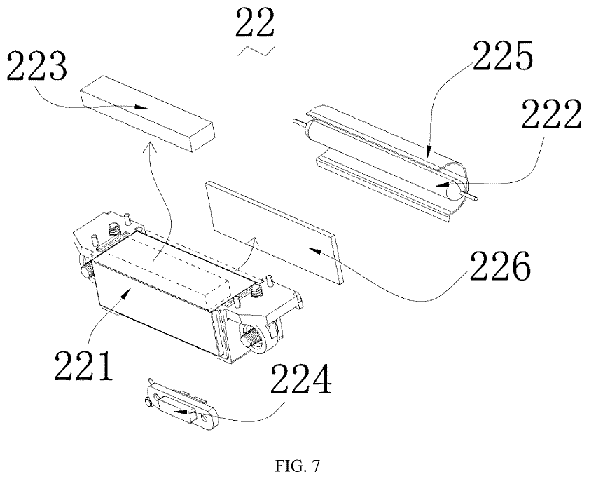

Referring to FIG. 7, the depilation mechanism 22 includes a cold compress portion 221, an emitter 222, a refrigeration element 223, a skin detection portion 224, a reflecting plate 225, an insulating plate 226, and a mounting component (not marked).

Both the emitter 222 and the refrigeration element 223 are electrically connected to the circuit apparatus 3, the cold compress portion 221 is clamped to the mounting component, the refrigeration element 223 is tightly attached to the cold compress portion 221 to cool the cold compress portion 221, the insulating plate 226 is located between the cold compress portion 221 and the emitter 222 to prevent heat of the emitter 222 from being transferred to the cold compress portion 221, and the reflecting plate 225 is disposed on one side of the emitter 222 away from the cold compress portion 221, so that light emitted by the emitter 222 is concentrated on the cold compress portion 221.

Further, the cold compress portion 221 is made of a crystal material, and specifically may be made of sapphire, K9 glass, or crystal glass, provided that a transparent crystal material is met. Optionally, the cold compress portion is made of a sapphire material.

It may be understood that, because the cold compress portion 221 is made of the sapphire, the cold compress portion 221 may be used as a light outlet. When the emitter 222 emits the light, because the sapphire has a relatively strong heat conductive property, the refrigeration element 223 and the cold compress portion 221 tightly attached to a surface of the emitter can efficiently generate a heat exchange, to achieve an optimal refrigeration effect. The cold compress portion 221 made of the sapphire may be round or rectangular, which is not limited herein. A surface of the cold compress portion 221 away from the emitter 222 is in contact with the human body, and a contact surface may be an arc surface or a flat surface, and is preferably a flat surface.

Further, the emitter 222 may be an IPL tube, and is located on one side of the cold compress portion 221 away from contact with the human body. The light emitted by the emitter 222 passes through the cold compress portion 221, and is emitted into skin of a user. A light color emitted by the emitter is not limited, the light may be a colored light, a composite light, or the like, and a specific wavelength and a frequency are determined according to a use situation.

Further, the skin detection portion 224 is electrically connected to the circuit apparatus 3 by using a capacitance touch detection principle. When the cold compress portion 221 is in contact with the skin, an internal preset capacitance detection apparatus detects whether a machine is actually in contact with the skin, to reduce a security problem caused by misoperations of the user.

Furthermore, a shape of the reflecting plate 225 is not limited, provided that the light from the emitter 222 is concentrated in a direction of the cold compress portion 221. The reflecting plate may be optionally U-shaped, an opening of the reflecting plate faces the cold compress portion 221, and the emitter 222 is located at a center of the opening of the U-shaped reflecting plate 225. In addition to concentrating the light, the reflecting plate also prevents a temperature of the emitter 222 during working from being dissipated to other places.

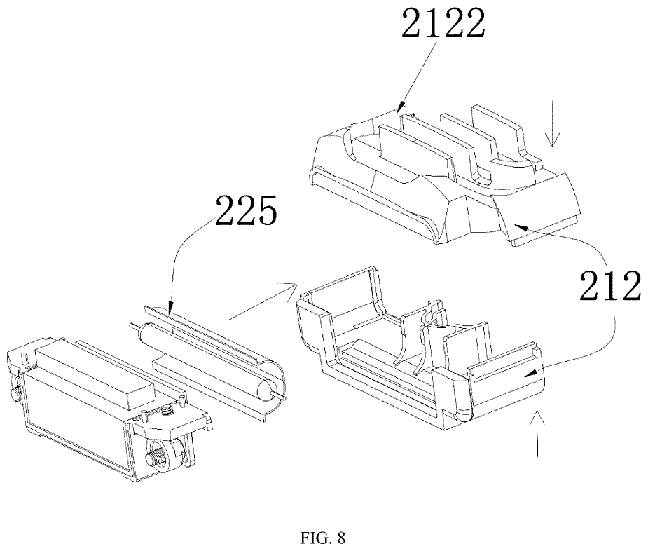

Referring to FIG. 8, the reflecting plate 225 is clamped to the heat conductive component 212, and is disposed on a surface away from the first vent 2121, so that the first vent 2121 forms a sealed channel.

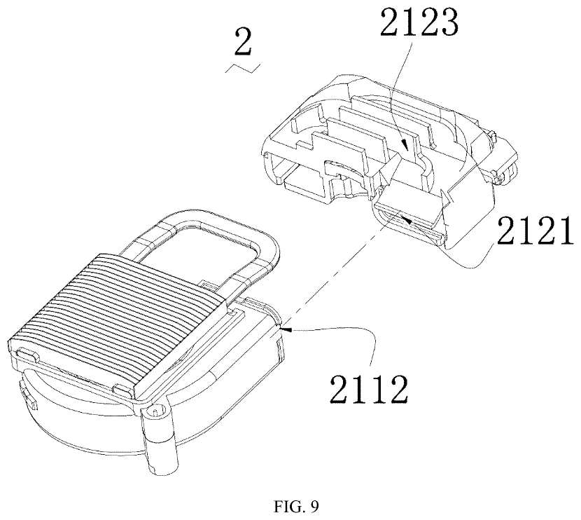

Referring to FIG. 8 and FIG. 9, when being connected, the fan 211 and the heat conductive component 212 are sleeved on the first vent 2121 through the fan air outlet 2112. Because a size a of the fan air outlet 2112 is greater than a size b of the first vent 2121, an extra gap c forms a second vent 2122, so that the air is divided into two directions in the first vent 2121 and the second vent 2122.

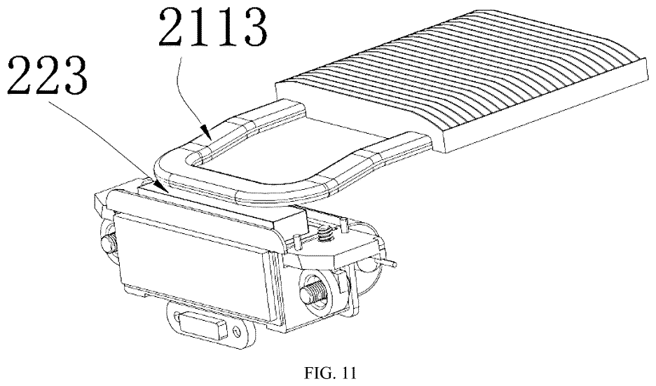

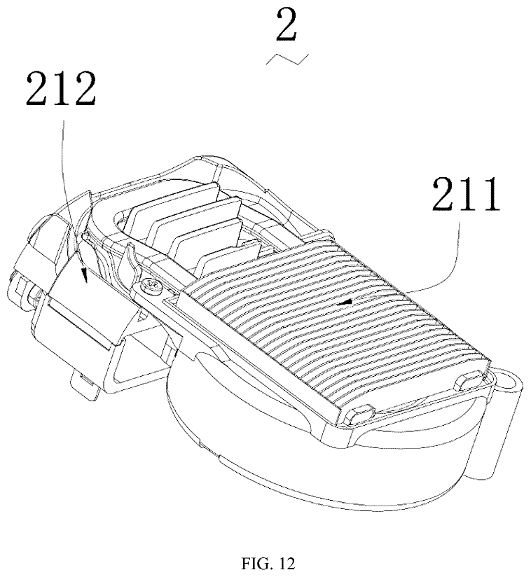

Referring to FIG. 11 and FIG. 12, further, when the fan 211 and the heat conductive component 212 are connected, the heat conductive element 2113 on the fan 211 is tightly attached to the refrigeration element 223, and the first vent 2121 is exposed on one side of the fan 211, to discharge the air sucked by the fan 211.

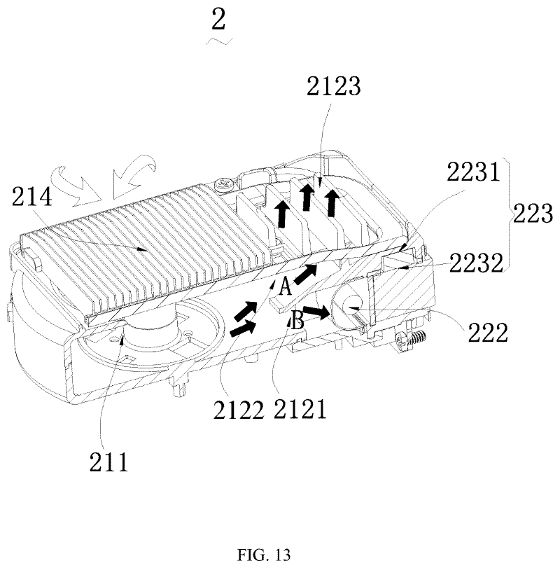

Referring to FIG. 11 and FIG. 13, the refrigeration element 223 is optionally a refrigeration element 223 in a semiconductor refrigeration manner, the refrigeration element 223 includes a heating surface 2231 and a refrigeration surface 2232 that are disposed opposite to each other, and the refrigeration surface 2232 of the refrigeration element 223 is tightly attached to the cold compress portion 221, to cool the cold compress portion 221.

It may be understood that, due to the particularity of the principle of the light depilation technology, a lot of heat is generated when the light is emitted, and when the cold compress portion 221 is in contact with the skin, to prevent the light from generating a burning pain to the user, a structure for temperature reduction needs to be disposed inside a body of the portable depilation instrument 100. Regardless of manners of temperature reduction, the heat is generated under the heat exchange, and the heating surface 2231 of the refrigeration element 223 is connected to the heat conductive element 2113. In addition to the refrigeration element 223, other internal elements, such as the emitter 222, and the power supply apparatus 4 and the circuit apparatus 3 inside the shell 1 all generate a lot of heat.

If the heat is not discharged in time, the refrigeration element 223 cannot achieve the refrigeration effect at maximum efficiency, thereby failing to reach a cooling temperature at which the skin is temporarily paralyzed.

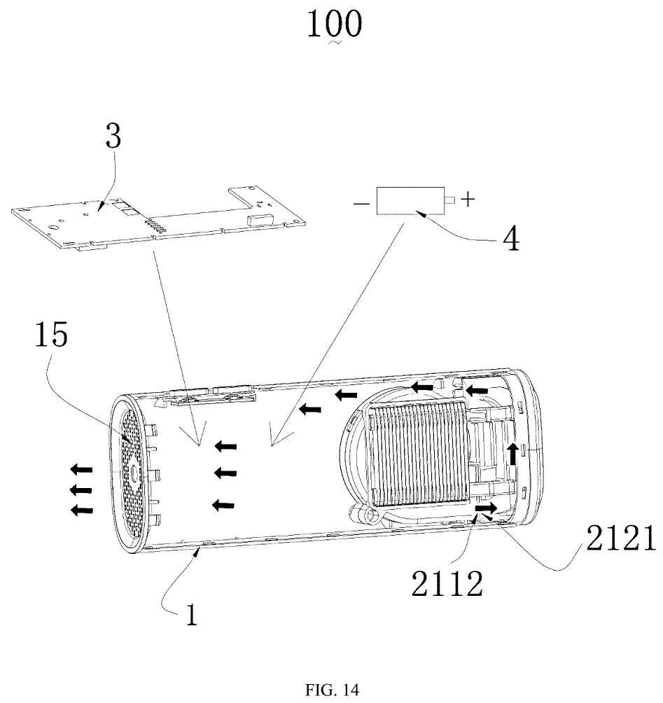

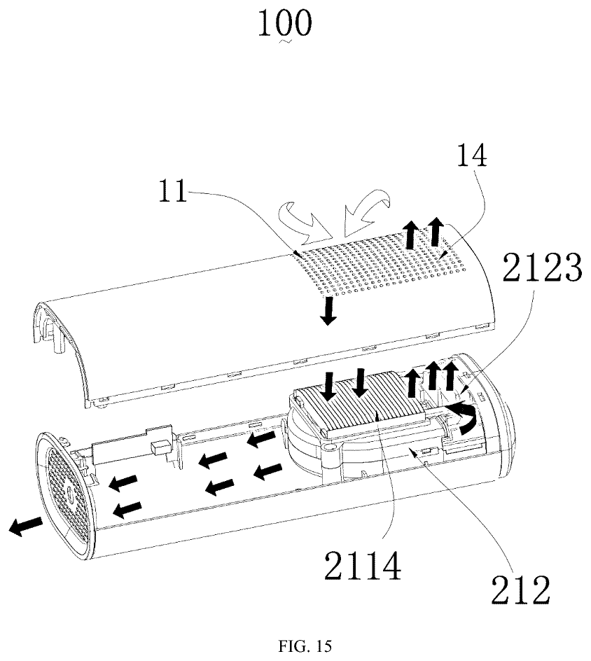

Referring to FIG. 13, FIG. 14, and FIG. 15, according to the foregoing two directions of the first vent 2121 and the second vent 2122 of the air, one of the directions is the first vent 2121, and the first vent 2121 is in communication with the first outlet 15 on the shell 1 to form a first channel A in which the air flows.

It may be understood that, the external air is sucked from the inlet 11. After entering the first vent 2121 through the second vent 2122, the external air passes through the emitter 222 to reduce a temperature of the emitter 222, then passes through the circuit apparatus 3 and the power supply apparatus 4 inside the shell 1, and is discharged from the first outlet 15 of the shell 1, to form an air path of the first channel, and temperature reduction and cooling are also performed on regions through which the air passes. For example, the circuit apparatus 3 and the power supply apparatus 4 are disposed in the region through which the air passes, that is, one side of inside of the shell 1 near the first outlet 15, to perform heat dissipation on the circuit apparatus 3 and the power supply apparatus 4 and bring the air out of the first outlet 15.

The heat conductive component 212 and the second heat dissipation fin 2123 at an upper end of the heat conductive component are integrated, an outer surface of the heat conductive component 212 and an inner wall of the shell 1 are enclosed to form a space, and a second vent 2122 is formed at the fan air inlet 2111 in an attachment manner. The second heat dissipation fin 2123 is disposed in the space and faces the second outlet 14, so that the air exits in a direction towards the second outlet 14, and therefore, the second vent 2122 and the second outlet 14 on the shell 1 form a second channel B.

It may be understood that, the second heat dissipation fin 2123 not only has a function of heat dissipation, but also has a function of guiding the air. Based on this, the second heat dissipation fin is designed as a fin structure, so that internal air may directly exit towards the second outlet 14. When passing through the second heat dissipation fin 2123, the air flows at the refrigeration element 223, to perform temperature reduction on the refrigeration element 223. The reason why the air is directly discharged is that the refrigeration element 223 is an element that generates a maximum amount of heat inside the portable depilation instrument 100, and therefore a path of the air in an air circulation needs to be reduced, so that the air is discharged at the first time, thereby achieving the cooling and temperature reduction effect at maximum efficiency.

Based on the first channel A and the second channel B, in some embodiments, a manner of heat dissipation is not using the fan 211 for air cooling, but an external cooling medium such as water or coolant may be used, provided that the external medium can bring heat of the two channels.

Based on the first channel A and the second channel B, in some embodiments, the heat dissipation mechanism 21 may not have the heat conductive element 2113 and the first heat dissipation fin 2114, and may perform heat dissipation on the emitter 222 and the refrigeration element 223 by directly using air of the fan 211 or another medium.

The second outlet 14 is disposed on one side of the inlet 11, both are disposed on a side surface of the shell 1 near one end of the heat conductive element 2113, and the first outlet 15 is located at an end portion of the shell 1 away from the heat conductive element 2113, so that air of the first channel is able to completely pass through inside of the entire shell 1, and an air inlet direction of the inlet 11 is parallel to an air outlet direction of the second outlet 14, and is perpendicular to an air outlet direction of the first outlet 15.

A contact end surface between the cold compress portion 221 and the skin is disposed on a same side as an end surface at which the skin detection portion 224 is exposed, so that when light depilation is performed on the skin, the cold compress portion 221 is in contact with a periphery of the irradiated skin, to perform cold compress and temperature reduction on the periphery of the irradiated skin, to reduce a burning sensation generated by the irradiated skin. In addition, the cold compress portion 221 may be close to zero temperature, to really make the skin near the light outlet reach infinitely close to a freezing point, so that the burning sensation of the skin can be alleviated, and short-time contact does not cause skin damage.

Under the guidance of the second heat dissipation fin 2123, air entering the second vent 2122 is directly discharged from the first outlet 15 after passing through the heat conductive element 2113. In this way, an air path of the second channel is formed, and similarly, temperature reduction and cooling are performed on the regions through which the air passes.

In the present invention, an improvement is made on a portable depilation instrument using an original heat dissipation mode. The fan 211 is used to suck external air, through the heat conductive component 212, an air direction separately flows to the first channel and the second channel, the first channel and the second channel are completely isolated, the refrigeration element 223 and the emitter 222 are respectively located in positions through which the first channel and the second channel pass, and as the air flows in the completely isolated first channel and second channel, heat dissipation is separately performed on the refrigeration element and the emitter; and the first channel may further perform temperature reduction on other heat generation elements inside the shell 1, for example, elements such as the circuit apparatus 3 and the power supply apparatus 4, to achieve an efficient and complete temperature reduction and cooling effect.

In some embodiments, a plurality of channels more than the two channels in this embodiment are provided. Except the refrigeration element 223 and the emitter 222, a plurality of channels may be alternatively provided, and other heat generation elements are alternatively disposed in remaining channels. This falls within the protection scope of the present invention.

In some embodiments, a portable depilation instrument is provided with a light-emitting body (not shown in the figure), and structural features described in all the foregoing embodiments are used.

### Embodiment 2

To better resolve the foregoing problem of incomplete heat dissipation, the present invention further provides a second embodiment. The second embodiment is an extension on the basis of the portable depilation instrument 100 in the first embodiment, and includes all features of the portable depilation instrument 100 in the first embodiment.

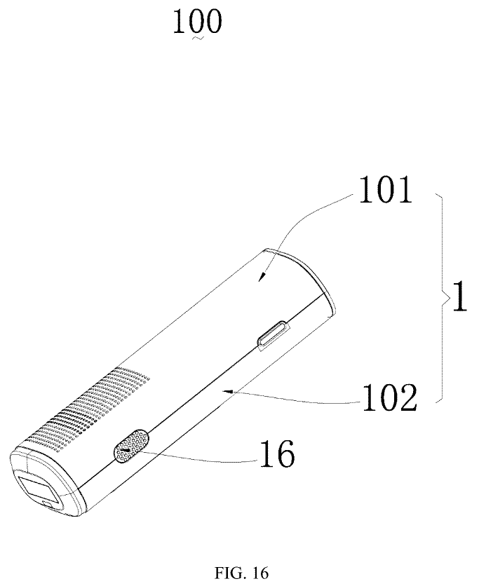

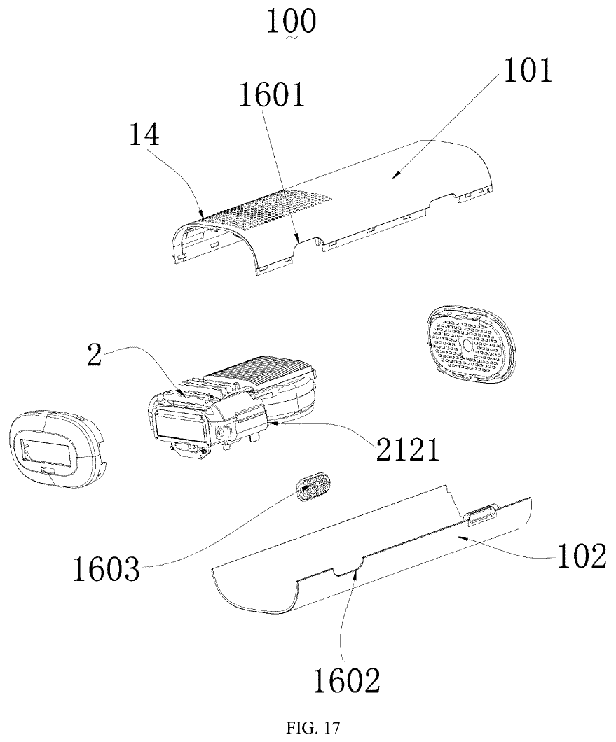

Referring to FIG. 16 and FIG. 17, the portable depilation instrument 100 provided in the second embodiment is provided with a third outlet 16 on the shell 1, and the third outlet 16 is optionally disposed on a peripheral side of the first vent 2121, and is in communication with a cavity inside the shell 1, so that the external cooling medium from the first vent 2121 may be discharged from the third outlet 16 on the peripheral side of the shell 1.

Further, in the present invention, optionally, the upper shell 101 and the lower shell 102 are respectively disposed on a first groove 1601 and a second groove 1602, an exhaust structure 1603 capable of being in communication with the inside of the shell 1 is disposed between the first groove 1601 and the second groove 1602, and after the upper shell 101 and the lower shell 102 are connected, a gap is formed between the first groove 1601 and the second groove 1602, and the exhaust structure 1603 is disposed in the gap to cause the cavity inside the shell 1 to be in communication with outside, to form a third outlet 16.

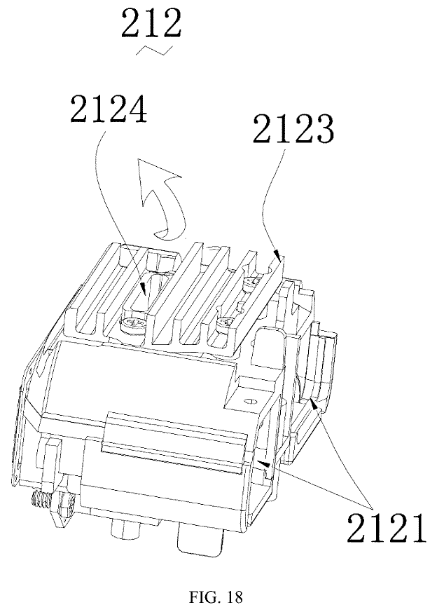

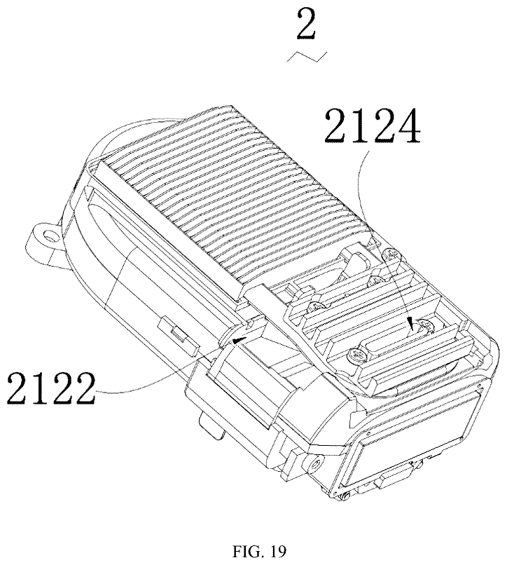

Referring to FIG. 18 and FIG. 19, furthermore, the heat conductive component 212 is provided with a third vent 2124 at the second heat dissipation fin 2123, the third vent 2124 faces a guiding direction of the second heat dissipation fin 2123, that is, faces a direction of the second outlet 14, and the third vent 2124 is in communication with the first vent 2121, so that the external cooling medium is discharged through the third vent 2124 in a direction from the second heat dissipation fin 2123 to the second outlet 14 after passing through the first vent 2121, to better speed up a progress of the heat exchange, thereby achieving a better cooling effect on the depilation mechanism 2.

The third vent 2124 is only a means of implementing the communication of the first vent 2121, and a quantity and a communication manner are not limited. That is, a fourth or fifth vent may be alternatively provided, or the heat conductive component 212 is correspondingly provided with at least three through holes provided on a surface of the second heat dissipation fin 2123, so that the vent is in communication with the first vent 2121, provided that the external cooling medium that flows inside the first vent 2121 is discharged through the second heat dissipation fin 2123.

Therefore, through the first vent 2121 and the second vent 2122, and under the action of the first outlet, the second outlet, the third outlet (not shown in the figure), the first vent 21, the second vent 2122, and the third vent 2124, the cooling efficiency is further improved, so that better ice feeling experience is brought to the user.

As summarized in Embodiment 2, the outlets disposed on the shell 1 and the vents on the depilation mechanism 2 are mainly described in this embodiment. Both a quantity of the outlets and a quantity of the vents are not limited. For example, the third outlet 16 is mainly used for discharging the external cooling medium passing through the first vent 2121 to the outside of the shell 1, or the third vent 2124 is mainly used for discharging the external cooling medium passing through the first vent 2121 to the outside of the shell 1 through the second heat dissipation fin 2123.

A person skilled in the art should also understand that if all or a part of the components of the structure in the present invention are combined in a manner of fusion, physical connection, or the like, for example, positions of the components in the depilation apparatus 2 are moved; or the components are integrated; or the components are detachably designed; and a replacement of a feature quantity and a change in feature morphology that is not used as a function are made, all combined components may form a device/apparatus with a specific function, and such a device/apparatus replaces a corresponding component in the present invention, and also falls within the protection scope of the present invention.

Compared with the prior art, the portable depilation instrument provided in the present invention has the following advantages. To help users hold, all the household portable depilation instruments are designed relatively small. Therefore, due to accumulation of a large amount of heat, heat dissipation is a very important problem, and if the heat dissipation is not good, a pain may be brought to the users during depilation, and even a security problem is caused. Previously, only a single inlet and a single outlet are provided in the portable depilation instrument, to form a channel for heat dissipation. However, it is not only a single element that generates heat inside the portable depilation instrument. However, if a plurality of heat generation elements rely on a single channel formed by one inlet and one outlet for heat dissipation, heat generated by the heat generation elements affect each other, resulting in a poor heat dissipation effect, and a working environment of the heat generation elements in the channel is affected. For example, the refrigeration element and the emitter that both generate heat are placed in a same heat dissipation channel. When passing through one of the refrigeration element and the emitter, the external cooling medium brings the heat to another place. Therefore, in the present invention, a plurality of heat dissipation channels are provided in the portable depilation instrument, and different heat generation elements may be disposed in different heat dissipation channels. The heat dissipation channel may be one inlet corresponding to a plurality of outlets, or may be a plurality of inlets corresponding to one outlet. Therefore, under the action of the heat conductive component, the external cooling medium may cool a plurality of heat generation elements inside the shell of the portable depilation instrument without interfering with each other.

The foregoing descriptions are only preferred embodiments of the present invention, but are not intended to limit the present invention. Any modification, equivalent replacement, or improvement made without departing from the principle of the present invention shall fall within the protection scope of the present disclosure.

What is claimed is:

1. A portable depilation instrument, comprising:
a shell, defining an accommodating space;
a cold compress portion, exposed from the shell and configured to contact skin of a user; and
an emitter, arranged in the accommodating space and located on a side of the cold compress portion facing away from the skin of the user, and the emitter being configured to emit light that irradiates the skin of the user;
wherein, the portable depilation instrument defines a first channel and a second channel inside the shell, allowing external cooling medium entering the shell to separately flow through the first channel to cool the emitter and flow through the second channel to cool the cold compress portion.

2. The portable depilation instrument of claim 1, wherein the first channel and the second channel are isolated.

3. The portable depilation instrument of claim 1, wherein the cold compress portion is made of a light-transmitting material and disposed on a light output path of the emitter, and the light emitted by the emitter passes through the cold compress portion.

4. The portable depilation instrument of claim 1, further comprising:
a heat dissipation mechanism arranged in the accommodating space and thermally coupled to the cold compress portion for cooling the cold compress portion.

5. The portable depilation instrument of claim 4, wherein at least part of the heat dissipation mechanism is located in the second channel.

6. The portable depilation instrument of claim 4, wherein the heat dissipation mechanism comprises:
a heat conductive element, one end of the heat conductive element being thermally coupled to the cold compress portion.

7. The portable depilation instrument of claim 6, wherein part of the heat conductive element is located in the second channel.

8. The portable depilation instrument of claim 1, wherein the emitter is located in the first channel.

9. The portable depilation instrument of claim 6, further comprising:
a refrigeration element having a refrigeration surface and a heating surface;
wherein the refrigeration surface is attached to the cold compress portion to cool the cold compress portion, and the heating surface is connected to the heat conductive element to dissipate heat.

10. The portable depilation instrument of claim 6, wherein the heat dissipation mechanism further comprises:
a first heat dissipation fin disposed at another end of the heat conductive element.

11. The portable depilation instrument of claim 10, wherein the shell is defined with an inlet, the external cooling medium comprises air, and the heat dissipation mechanism further comprises:
a fan, defined with a fan air inlet facing the inlet of the shell;
wherein the first heat dissipation fin is disposed between the fan air inlet and the inlet of the shell.

12. The portable depilation instrument of claim 1, wherein
the external cooling medium comprises air;
the shell is defined with an inlet, a first outlet communicated with the first channel, and a second outlet communicated with the second channel;
the air entering the shell through the inlet is configured to separately flow through the first channel and leave the shell through the first outlet, and flow through the second channel and leave the shell through the second outlet.

13. The portable depilation instrument of claim 12, further comprising:
a heat conductive component, configured to guide the external cooling medium to separately pass through the first channel and the second channel.

14. The portable depilation instrument of claim 13, wherein the heat conductive component defines a part of the first channel, and the shell and one side of the heat conductive component facing the second outlet cooperatively define a part of the second channel.

15. The portable depilation instrument of claim 13, wherein
the external cooling medium comprises air;
the heat conductive component is defined with a first vent and a second vent;
the inlet, the first vent, and the first outlet are sequentially in communication with each other to form the first channel; and
the inlet, the second vent, and the second outlet are sequentially in communication with each other to form the second channel.

16. The portable depilation instrument of claim 15, wherein a surface of the heat conductive component facing the second outlet is provided with a second heat dissipation fin.

17. The portable depilation instrument of claim 15, wherein
the first outlet is located at an end portion of the shell away from the emitter, to allow the air passed the first channel to further passes through the interior of the shell from one end to the other end, and carry heat out of the interior of the shell;
the second outlet is located on one side of the inlet, and the second outlet and the inlet are both located on a side surface of the shell near one end of the heat conductive element; and
an air inlet direction of the inlet is parallel to an air outlet direction of the second outlet, and the air inlet direction of the inlet is perpendicular to an air outlet direction of the first outlet.

18. The portable depilation instrument of claim 15, wherein the shell is further defined with a third outlet, and the third outlet is defined on a side surface of the shell or defined on a surface of the shell facing away from the second outlet.

19. The portable depilation instrument of claim 18, wherein the shell comprises an upper shell and a lower shell, the upper shell is defined with a first groove, and the lower shell is defined with a second groove, an exhaust structure communicated with an interior of the shell is provided between the first groove and the second groove, and in a case that the upper shell and the lower shell are connected, a gap is formed between the first groove and the second groove, and the exhaust structure is disposed in the gap to cause a communication between interior cavity of the shell and an outside, to form the third outlet.

20. The portable depilation instrument of claim 16, wherein the heat conductive component is further defined with a third vent at the second heat dissipation fin, the third vent faces a heat guiding direction of the second heat dissipation fin, and the third vent is in communication with the first vent.
